(19) United States
(12) Plant Patent Application Publication
(10) Pub. No.: US 2013/0340138 P1
(43) Pub. Date: Dec. 19, 2013

(54) Campanula plant named 'PKMTAK2'

(50) ;

(71) Applicant: Christian Hald Madsen, Korsor (DK)

(72) Inventor: Christian Hald Madsen, Korsor (DK)

(73) Assignee: Gartneriet PKM A/S

(21) Appl. No.: 13/507,208

(22) Filed: Jun. 13, 2012

(51) Int. Cl. A01H 5/00

(52) U.S. Cl. PLT/414

(57) ABSTRACT

A new and distinct cultivar of Campanula plant named ‘PKMTAK2’, characterized by its compact plant habit; dense and bushy plant form; vigorous growth habit; and large, white flowers.

### LATIN NAME OF GENUS AND SPECIES OF THE PLANT CLAIMED

Campanula takesimana

### VARIETY DENOMINATION

‘PKMTAK2’

### BACKGROUND OF THE INVENTION

The present invention relates to a new and distinct cultivar of Campanula plant, botanically known as Campanula takesimana, commonly known as Bellflower, and hereinafter referred to by the name ‘PKMTAK2’.

The new Campanula, ‘PKMTAK2’, is a product of a planned breeding program conducted by the inventor, Christian Hald Madsen, in Søhus, Denmark. The new Campanula originated from a cross made by the inventor in March 2009 between a proprietary selection of Campanula takesimana designated 21.01.0083 (breeder reference, unpatented) as the female parent and a proprietary selection of Campanula punctata designated 39.00.3010 (breeder reference, unpatented) as the male parent. The new Campanula ‘PKMTAK2’ was discovered and selected by the inventor as a single flowering plant within the progeny of the stated cross in a controlled environment in Søhus, Denmark. The inventor selected ‘PKMTAK2’ on the basis of its compact plant habit and white flowers.

Asexual reproduction of the new Campanula cultivar by terminal cuttings has demonstrated that the combination of characteristics as herein disclosed for the new cultivar are firmly fixed and retained through successive generations of asexual reproduction. The new cultivar reproduces true-to-type.

### SUMMARY OF THE INVENTION

The following traits have been repeatedly observed and are determined to be the unique characteristics of ‘PKMTAK2’. These characteristics in combination distinguish ‘PKMTAK2’ as a new and distinct cultivar:
1. White flowers;
2. Vigorous growth habit, but no need for chemical growth retardation.

Plants of the cultivar ‘PKMTAK2’ can be compared to the Campanula takesimana cultivar ‘PKMTAK1’ (patented, U.S. Plant Pat. No. 19,412). In side-by-side comparisons conducted by the inventor in Stige, Denmark, plants of the instant cultivar ‘PKMTAK2’ and plants of the cultivar ‘PKMTAK1’ differ in the following characteristics:
1. Plants of the ‘PKMTAK2’ have white flowers whereas plants of ‘PKMTAK1’ have red-purple flowers.
2. Plants of ‘PKMTAK2’ have green stems whereas plants of ‘PKMTAK1’ have greyed-orange stems.

### BRIEF DESCRIPTION OF THE PHOTOGRAPHS

The accompanying colored photographs illustrate the overall appearance of the new Campanula ‘PKMTAK2’, showing the colors as true as it is reasonably possible to obtain in colored reproductions of this type. Colors in the photographs may differ slightly from the color values cited in the detailed botanical description, which more accurately describe the actual colors of ‘PKMTAK2’.

FIG. 1 comprises a side perspective view of a typical flowering plant of PKMTAK2.

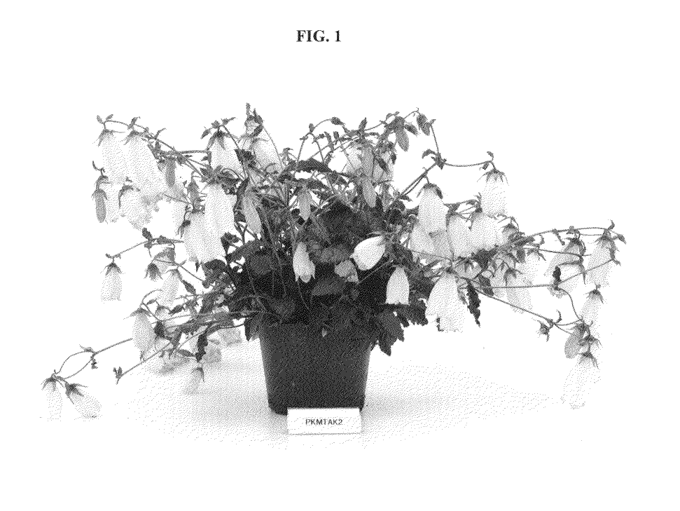

FIG. 2 comprises a close-up view of a typical mature bud of PKMTAK2.

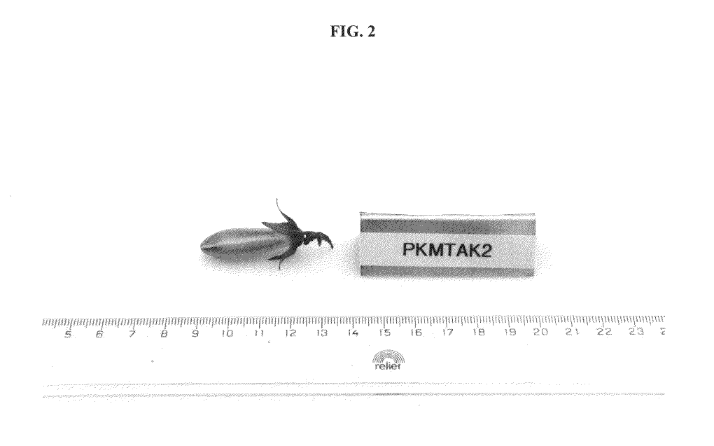

FIG. 3 comprises a close-up view of a typical young bud of PKMTAK2.

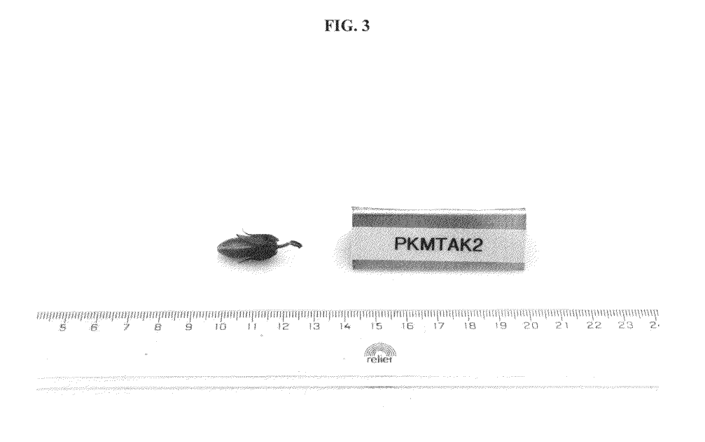

FIG. 4 comprises a side perspective view of a typical flower of PKMTAK2.

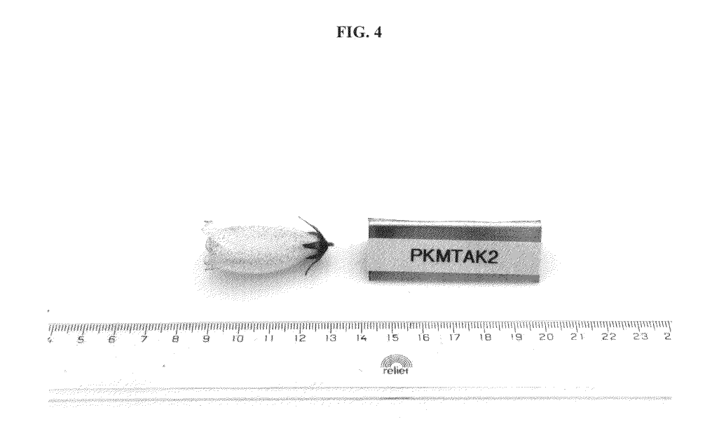

FIG. 5 comprises a front perspective view of a typical flower of PKMTAK2.

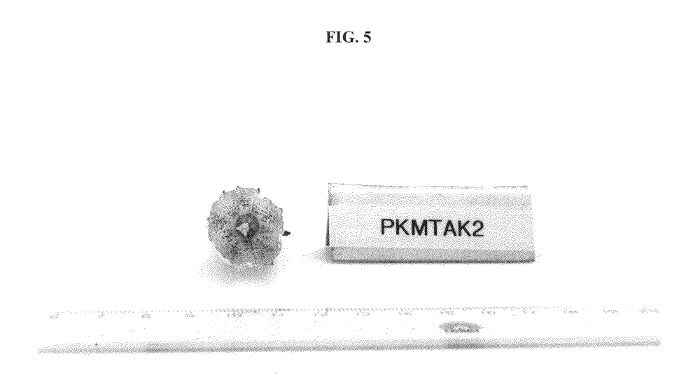

FIG. 6 comprises a close-up view of a typical apical leaf of PKMTAK2.

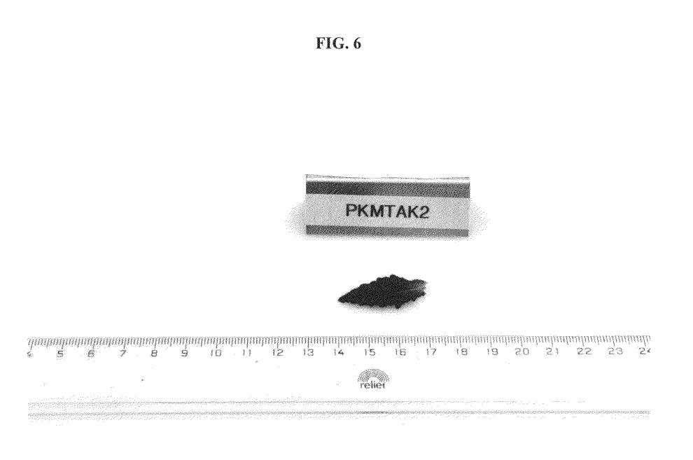

FIG. 7 comprises a close-up view of a typical basal leaf of PKMTAK2.

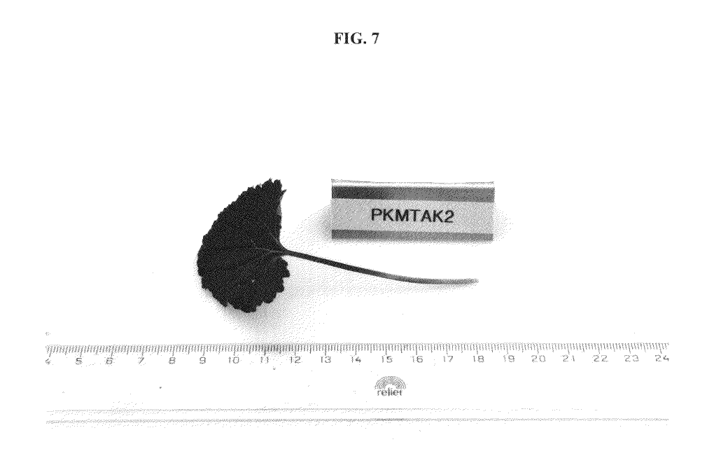

### DETAILED BOTANICAL DESCRIPTION

The new Campanula ‘PKMTAK2’ has not been observed under all possible environmental conditions. The phenotype of the new cultivar may vary with variations in environment such as temperature, light intensity, day length, and fertility level without any variance in genotype.

The aforementioned photographs, together with the following observations, measurements and values describe plants of the new Campanula ‘PKMTAK2’ as grown under greenhouse conditions. No growth retardants were used.

The age of the ‘PKMTAK2’ plants described is 10 months after cutting. Color references are made to The Royal Horticultural Society Colour Chart (R.H.S.), 5^{th} edition, except where general colors of ordinary significance are used.
Classification:
   Botanical.—Campanula takesimana.
Parentage:
   Female or seed parent.—Campanula takesimana designated 21.01.0083 (breeder reference, unpatented).
   Male or pollen parent.—Campanula punctata designated 39.00.3010 (breeder reference, unpatented).
Propagation:
   Type cutting.—Terminal vegetative cuttings.
   Time to initiate roots.—About 12 to 15 days at 18 to 21° C. in tunnels in a greenhouse.
   Root description.—Fine, well branched.
Plant description:
   Form.—Perennial, herbaceous plant with upright, compact plant habit. Produced as potted plant. Campanulate flowers in racemes. Freely branching from rhizomes.
   Crop time.—After rooting, about 9-11 months are required to produce finished flowering plants in 11 cm pots.
   Plant height (from pot rim to top of plant plane).—About 25 cm to 30 cm.
   Plant spread (width).—About 35 cm to 40 cm.
   Lateral branches.—Quantity: About 20 to 28 per plant. Length (including flowers): 14 cm to 27 cm. Diameter: 1 mm to 2 mm. Internode Length: About 30 mm to 90 mm.
   Stem.—Shape: Square. Strength: Stiff. Aspect: Upright, a few spreading. Pubescence: very short hairs, hispidulous Color: RHS 138A, green; some base stems RHS 166A greyed-orange.
Foliage description:
   Arrangement.—Alternate, heterophyllous.
   Basal leaves.—Overall shape: Cordate. Apex shape: Cuspidate to rounded. Base shape: Cordate. Length: 20-40 mm. Width: 20-50 mm. Margin: Crenate. Texture (both sides): Grooved.
   Apical (stem) leaves.—Overall shape: Lanceolate. Apex shape: Acute. Base shape: Equilateral. Length: About 25-40 mm. Width: About 12-18 mm. Margin: Serrate. Texture (both sides): Grooved.
   Pubescence.—Barbellulate along veins and margins, underside.
   Basal & apical leaves.—Mature: Upper surface: Green, RHS 137A. Lower Surface: Green, RHS 138C. Immature: Upper surface: Green, RHS 137B. Lower Surface: Green, RHS 137C. Venation: Pattern: Reticulate. Color (upper and lower surface): Green, 137C Petiole: Basal Length: 3 cm to 6 cm. Apical Length: Up to 1 cm. Diameter: 0-2 mm. Color: RHS 138A, green; some RHS 166A greyed-orange. Blotchy, angular.
Inflorescence description:
   Flower arrangement and shape.—Single, large, campanulate flowers in racemes, drooping, acropetal flowering habit.
   Natural flowering season.—May to July.
   Time to flower.—About 8 weeks after planting of rooted cutting.
   Flower longevity.—Longevity of individual flowers is highly dependent on temperature and light conditions. Flowers persistent.
   Inflorescence size.—Height: 10 to 12 cm.
   Number of flowers per inflorescence.—3 to 5.
   Flowers.—Arrangement and shape: Campanulate, acropetal, drooping. Flowers single, sympetalous, campanulate, somewhat gibbous. Height: 40 mm Diameter: 20 mm.
   Buds.—Length: Up to 30 mm. Diameter: Up to 10 mm. Shape: Oblong, ridged, fluted. Color: Young buds: RHS N144D yellow-green Mature buds: RHS 157A, green-white.
   Petals.—Appearance: Dull, single, sympetalous, sympetalous, somewhat gibbous. Quantity per flower: 5. Length: 7 mm Width: Base of lobe 10 mm. Overall Shape: Lobes triangular. Tip: Acute. Base: Fused. Margin: Entire. Texture: Glabrous.
   Color (when opening & when fully opened).—Outside bell: Ground color RHS N155C, white with dots of RHS 70A, red-purple. Inside bell: Ground color RHS N155C, white with shadows reflecting from dots outside bell. Fading: Fades to RHS 160D, greyed-yellow with dots RHS 70A, red-purple both inside and outside bell.
   Sepals.—Arrangement: Shiny and free. Quantity per flower: 5. Length: 15 mm. Width: 5 mm. Overall shape: Hastate, sessile, clasping. Tip: Caudate. Base: Sessile. Margin: Entire. Texture: Pubescent, very short, stiff hairs. Color (mature and immature): Upper and lower surfaces: Green, RHS 138A.
   Peduncles.—Length: About 10-14 mm. Diameter: About 1 mm Strength: Average. Angle: from 45 to 180° Color: RHS 166A, greyed-orange.
   Pedicels.—None.
Reproductive organs:
   Androecium.—Stamen: Quantity: 5, adenate to corolla until pollen has shed. Anther: Shape: Lanceolate, antisepalous, introse, basifixed and two-celled Curling after shedding pollen. Length: About 1 mm, filament 4 mm Color: RHS 158B, yellow-white. Pollen: Amount: Average. Color: RHS 158B, yellow-white.
   Gynoecium.—Pistil: Quantity: 1. Length: About 20 mm to 23 mm. Stigma: Shape: tripartite. Color: RHS 155D, white. Style: Length: About 2 mm. Color: RHS 36B, red. Ovary: Color: RHS 157D, green-white.
Seed/fruit: None observed.
Root:
   Structure.—Fibrous with many rhizomes.
   Color.—RHS 156B, greyed-white.
Weather tolerance: Plants of the new Campanula have generally good tolerance to drought, rain and wind, with low temperature resistance to −20° C.

I claim:

1. A new and distinct cultivar of Campanula plant named ‘PKMTAK2’, as illustrated and described herein.
